(19) United States
(12) Patent Application Publication
(10) Pub. No.: US 2016/0081959 A1
(43) Pub. Date: Mar. 24, 2016

(54) METHOD OF USING ARGININE SILICATE INOSITOL COMPLEX FOR WOUND HEALING OR REPAIR

(71) Applicant: Glanbia plc, Carlsbad, CA (US)

(72) Inventors: Jeremy David Bartos, San Marcos, CA (US); Mayuresh Dilip Bedekar, Carlsbad, CA (US)

(73) Assignee: GLANBIA PLC, Carlsbad, CA (US)

(21) Appl. No.: 14/850,685

(22) Filed: Sep. 10, 2015

Related U.S. Application Data
(60) Provisional application No. 62/054,789, filed on Sep. 24, 2014.

(51) Int. Cl. A61K 31/198; A61K 31/047; A61K 45/06; A61K 9/00

(52) U.S. Cl. CPC A61K 31/198; A61K 9/0053; A61K 9/0014; A61K 9/0019; A61K 31/047; A61K 45/06

(57) ABSTRACT

A method for treating a wound or injury in an individual is provided, comprising the steps of administering to the individual afflicted with the wound or injury a composition including an effective amount of arginine silicate inositol (ASI), optionally in combination with another component, and a nutraceutically or pharmaceutically acceptable carrier.

This application claims the benefit of earlier filed U.S. Provisional application No. 62/054,789, filed on Sep. 24, 2014, which is hereby incorporated by reference herein.

### TECHNICAL FIELD

A method of using arginine silicate inositol (ASI) complex is provided, optionally in combination with one or more nutritional components, flavoring compounds, dietary supplements, or other edible components. In an embodiment, a nutritional composition includes ASI. In another embodiment, ASI is used in a method of treating wounds, i.e. wound healing or repair.

### BACKGROUND

Treatment of wounds and other acute injuries is one of the primary responsibilities taken on by first responders (e.g., police, firemen, military, EMTs, and all manner of emergency medical service (EMS) personnel) and medical staff (e.g., nurses, orderlies, military medics, triage or emergency room staff, physicians, and surgeons). Methods of treatment for wounds are a critical need often requiring immediate care. Many established methods of treatment are known, included standard first aid (field dressing and bandaging), administration of drugs or medicines, and surgical repair including stitching, grafting and bone-setting.

Apart from a human emergency scenario, wound treatment and/or wound healing are an important component of medical treatment for recovery after injury. Additionally, certain chronic or degenerative conditions may require constant vigilance with respect to treatment of wounds or lesions. Indeed, a key component of wound healing and/or repair involves regenerative processes including reestablishing homeostasis in the affected tissues (Wong, et al., “Wound Healing: A Paradigm for Regeneration,” Mayo Clin. Proc. (2013) 88(9):1022-1031).

Currently, researchers are taking a more holistic view of human health and nutrition. Thus, integration of good nutrition into the daily routine is one way of maintaining good immune balance, cardiac and muscular tone, and metabolic efficiency. For example, a human clinical study on the effects of 17 g/day of arginine on wound healing was performed on 30 elderly patients who sustained an experimental surgical injury. After two weeks supplemented patients demonstrated significantly greater hydroxyproline and protein accumulation at the wound site when compared to non-supplemented controls. Lymphocyte response was also elevated in the supplemented group, as was IGF-1 (Kirk, et al., Surgery (1993) 114(2): 155-160).

In another example of the potential for wound healing, arginine has been used to treat anal fissures (Gosselink et al., Dis. Colon Rectum (2005) 48(4):832-7).

However, arginine suffers from drawbacks including low bioavailability and an undesirable taste. Better treatments and/or products are needed for therapeutic and/or nutritional use that can replace arginine alone. One useful material is arginine silicate inositol (ASI) complex.

Therefore, if certain nutritional products containing ASI were able to promote wound healing, this would represent a contribution to the medical and nutritional arts.

### SUMMARY

A nutritional composition is provided comprising an effective amount of arginine silicate inositol (ASI), optionally in combination with another component, and a nutraceutically acceptable carrier.

Other optional components can include compounds selected from vasodilators, NO-precursors, or eNOS activators. Further optional components can include compounds selected from proline, hydroxyproline, IGF-1 activators, or polyamine precursors.

A method for treating a wound in an individual is provided, comprising the steps of administering to the individual in need of such treatment a composition including an effective amount of arginine silicate inositol (ASI), optionally in combination with another component, and a nutraceutically or pharmaceutically acceptable carrier. The present method can include oral/nutritional, intravenous, or topical treatment.

### BRIEF DESCRIPTION OF THE DRAWINGS

FIG. 1 depicts plasma concentration of arginine (μg/ml) over time as single dose oral pharmacokinetic results from testing in fasted male Sprague Dawley rates (equivalent dose of 500 mg/kg L-Arginine) for L-Arginine (free base), L-Arginine HCl, ResArgin, and Arginine silicate inositol (ASI).

FIG. 2 depicts percent change in concentration of plasma L-arginine in human subjects after a single dose of ASI, represented as mean value vs. time, relative to baseline.

### DETAILED DESCRIPTION

Wounds are injuries that break the skin or other body tissues. They include cuts, scrapes, scratches, burns, electrical wounds, bites, stings, incisions, amputations, and punctured skin. They often happen because of an accident, but surgery, sutures, and stitches also cause wounds.

As used herein, “wound healing” refers to a biologically-mediated process of repair or remodeling that may involve treatment of skin or other body tissues with a medicinal or nutritional composition. Such treatments as applied to the human body may include, but are not limited to, treatment of damage or insult to skin, tissue, and organs, wherein the affected area may be, for example, cutaneous, subcutaneous, visceral, circulatory, lymphatic, gastrointestinal, mucosal, skeletal, muscular, etc.

In one example, human skin is a remarkably plastic organ that sustains insult and injury throughout life. Its ability to expeditiously repair wounds is paramount to survival and is thought to be regulated by wound components such as differentiated cells, stem cells, cytokine networks, extracellular matrix, and mechanical forces. These intrinsic regenerative pathways are integrated across different skin compartments and are being elucidated on the cellular and molecular levels. Recent advances in bioengineering and nanotechnology have allowed researchers to manipulate these microenvironments in increasingly precise spatial and temporal scales, recapitulating key homeostatic cues that may drive regeneration.

As used herein, “arginine” is intended to mean L-arginine (Arg), a naturally occurring amino acid, with or without a salt counterion, available in purified form from Glanbia Nutritionals, Inc. (Carlsbad, Calif.).

It has now unexpectedly been found that arginine silicate inositol (ASI) complex may be used to make an edible nutritional composition that is effective for treating wounds, i.e., useful for wound healing or repair. It is believed that ASI-containing compositions are useful for treating wounds including, but not limited to, cuts, scrapes, scratches, burns, electrical wounds, bites, stings, incisions, amputations, anal fissures, punctured skin, and the like.

In addition, the arginine and silica from ASI are highly bioavailable meaning that less is needed to provide a similar effect seen from other arginine sources. Stated in another way, ASI contains about 40% by weight arginine. The combination of the components as conjugates (silicon at 8% and inositol at 25%) make it more easily absorbed. Administration of less arginine is needed to provide overall improvement in taste, independent of the use of sweeteners or other taste-masking agents. The edible nutritional compositions described herein are more palatable (i.e., better tasting) than compositions containing arginine alone.

In one preferred embodiment, an edible nutritional composition containing arginine silicate inositol (ASI) is used for treating wounds. It is believed that oral administration of the ASI-containing compositions can promote wound healing and/or repair.

Other suitable routes of administration are contemplated for treating wounds, including topical administration of a nutraceutical, cosmetic, or pharmaceutical composition containing ASI. Another suitable route of administration is parenteral including intravenous (IV) injection, or alternatively, subcutaneous injection, either at the wound site or distal thereto.

A commercially available, bioavailable form of arginine silicate inositol (ASI), is available in purified form as Nitrosigine® from Nutrition 21 (Purchase, N.Y.) and Glanbia Nutritionals, Inc. (Carlsbad, Calif.).

ASI contains arginine and silicate, both of which can contribute to the facilitation of wound healing. Arginine in particular regulates many metabolic and physiologic body functions and has many attributes that support wound repair. In particular, arginine is nitrogen-rich. The average amino acid is 16% nitrogen by weight while L-arginine is 32% nitrogen by weight (molar mass). Arginine is also the only substrate for nitric oxide synthesis. Nitric oxide has a beneficial effect on circulatory status and increases blood supply to the wound. Nitric oxide has also been shown to have a beneficial effect in muscle repair after acute or chronic injuries. In addition, arginine is a precursor to proline, which is converted to hydroxyproline and then to collagen which would help facilitate healing of skin and connective tissues, as well as polyamines, which are the cell's building blocks and integral for wound repair. Arginine can also be broken down by arginase into ornithine, which is another main component of collagen. Arginine also has a positive influence on the body's levels of insulin-like growth factor-I (IGF-I), a hormone that promotes wound healing. Finally, arginine is known to contain immune-enhancing properties that reduce the risk of infectious complications of a wound.

The Kirk, et al. (1993) study discussed above used arginine as a therapeutic agent for wound healing. There are several issues with using arginine or an arginine salt for products particularly geared towards clinical nutrition. The first is that arginine is not very bioavailable; it takes ca. 5 grams of arginine base or arginine HCl or ca. 3 grams of arginine alpha ketoglutarate (AAKG) to induce a significant vasodilatory response. In contrast, similar responses have been shown with only 750 mg of ASI, presumably due to the increased bioavailability of the arginine coming from the ASI. In addition, arginine is known to have a very unpleasant taste which makes it relatively unpalatable, especially for patients coming off of surgery who may be suffering from nausea or who have other wound healing needs. The smaller dose of ASI compared to other forms of arginine may help make a product more palatable or may be easier to mask flavor-wise.

Silicate is also a component of ASI and may also contribute to wound healing on its own. As used herein, “silicate” means any of a number of materials including, but not limited to silica, silica-containing compounds, silicate salts, esters, and the like. Useful cationic counterions for silicate include, but are not limited to, sodium, potassium, lithium, calcium, magnesium, and the like. Silica is well known to support wound healing because it is essential in collagen production and connective tissue formation. As such, silica stimulates the rapid regrowth of damaged skin tissue. In fact, silica is now being used topically as a component of dressings for the rapid healing of open wounds. A study in mice demonstrated that ingested silica was also effective at helping to close and heal wounds (Oberbaum, et al., Harefuah (1992) 123(3-4):79-82, 156). In addition, studies have shown that silica deprivation leads to longer recovery times and less efficient healing (Seaborn, et al., Biol. Trace Elem. Res. (2002) 89(3):251-61). Horsetail, a plant known to be high in silica, has traditionally been used to heal burns and minor wounds as well.

Single Dose Oral Pharmacokinetic (PK) Study

Oral PK was determined in rats for ASI and several comparative compounds. The objective of the study was to perform single dose oral pharmacokinetic study of a conjugate preparation of a 1:1 molar combination of L-arginine and resveratrol (ResArgin), L-Arginine base, L-Arginine hydrochloride, and arginine silicate inositol (ASI) in male Sprague Dawley rats at a dose equivalent to 500 mg L-Arginine/kg body weight.

ResArgin is a conjugate preparation of a 1:1 molar combination of L-arginine and resveratrol reacted in ethanol at 80-85° C., then water added at the same temperature, which is concentrated and isolated as an amorphous solid powder (available from Anthem Biosciences, Bangalore, India).

L-Arginine base (99% purity) is available from Glanbia Nutritionals, Inc. (Carlsbad, Calif.).

Arginine silicate inositol (ASI) as Nitrosigine® is commercially available as described above having a content of 449 mg L-Arginine/gram.

Vehicle for oral route of administration: 0.5% carboxymethylcellulose (CMC) in sterile water.

Test system: Sprague Dawley rats, male. Aged 7-9 weeks at the time of receipt.

Sample size: Study groups—4 groups per study; 5 animals/group.

Study procedure: Test compound oral formulations and animal preparation.

Oral formulation for ResArgin. About 2.09 grams of ResArgin was weighed and transferred into a clean mortar. Using a pestle with drop by drop addition of 0.5% Carboxymethylcellulose in sterile water, the test item was triturated until it formed a fine paste. Then some more volume of 0.5% Carboxymethylcellulose solution was added with continuous trituration to get a uniform suspension. The formulation from the mortar was transferred completely into a graduated container and the volume of the suspension was made up to 18 mL using 0.5% Carboxymethylcellulose in sterile water.

Oral formulation for Arginine base. About 0.91 grams of L-Arginine Base was weighed and transferred into a clean mortar. Using a pestle with drop by drop addition of 0.5% Carboxymethylcellulose in sterile water, the test item was triturated until it formed a fine paste. Then some more volume of 0.5% Carboxymethylcellulose solution was added with continuous trituration to get a uniform suspension. The formulation from the mortar was transferred completely into a graduated container and the volume of the suspension was made up to 18 mL using 0.5% Carboxymethylcellulose in sterile water.

Oral formulation for Arginine HCl. About 1.09 grams of L-Arginine HCl was weighed and transferred into a clean mortar. Using a pestle with drop by drop addition of 0.5% Carboxymethylcellulose in sterile water, the test item was triturated until it formed a fine paste. Then some more volume of 0.5% Carboxymethylcellulose solution was added with continuous trituration to get a uniform suspension. The formulation from the mortar was transferred completely into a graduated container and the volume of the suspension was made up to 18 mL using 0.5% Carboxymethylcellulose in sterile water.

Oral formulation for Arginine silicate inositol (ASI). About 2.00 grams of ASI was weighed and transferred into a clean mortar. Using a pestle with drop by drop addition of 0.5% Carboxymethylcellulose in sterile water, the test item was triturated until it formed a fine paste. Then some more volume of 0.5% Carboxymethylcellulose solution was added with continuous trituration to get a uniform suspension. The formulation from the mortar was transferred completely into a graduated container and the volume of the suspension was made up to 18 mL using 0.5% Carboxymethylcellulose in sterile water.

Animal Husbandry

A. Conditions: Animals were housed under standard laboratory conditions, in an environmentally monitored air-conditioned room with adequate fresh air supply (10-15 Air changes per hour), room temperature 22±3° C. and relative humidity 30-70%, with 12 hours light and 12 hours dark cycle. The temperature and relative humidity were recorded once daily.

B. Housing: A maximum of three animals were housed in a standard polypropylene cage (Size: L 290×B 220×H 140 mm) with a stainless steel mesh top grill having facilities for holding pelleted food and drinking water in a water bottle fitted with stainless steel sipper tube. Clean sterilized corncob was provided as bedding material.

C. Feed: The animals were fed ad libitum throughout the acclimatization and experimental period except for a period of fasting. Nutrilab rodent feed (Manufactured by Provimi Animal Nutrition Pvt Ltd) was provided.

D. Water: Water was provided ad libitum throughout the acclimatization and experimental period. Water is from Aqua guard water filter cum purifier, which will be autoclaved and provided in polypropylene water bottles with stainless steel sipper tubes.

Acclimatization

The rats were acclimatized for a minimum period of five days to experimental room conditions and observed for clinical signs daily. Veterinary examination of all the animals were performed on the day of receipt, daily and on the day of randomization.

Study Grouping

The animals for the experiment were weighed and arranged in ascending order of their body weights. These stratified body weight of the rats were distributed to all the experimental groups, such that body weight variation of animals selected for the study did not exceed ±20% of the mean body weight. The grouping was done one day prior to the initiation of treatment. Body weights of the animals were analyzed statistically for mean body weight to rule out the statistical significant difference between groups.

Study Dosing

TABLE 1

| | | Dose | | | |
|---|---|---|---|---|---|
| | | (mg) | Dose | | |
| | | (Equivalent to | Volume | | No. of |
| Study | Test | 500 mg L-Arg | (mL/kg | Conc. | Animals/ |
| Group | compound | Base/kg)* | b.w.) | (mg/mL) | Group |
| 1 | ResArgin | 1162.02 | 10 | 116.21 | 5 |
| 2 | L-Arginine | 503.52 | 10 | 50.35 | 5 |
| | Base | | | | |
| 3 | L-Arginine | 604.65 | 10 | 60.46 | 5 |
| | HCl | | | | |
| 4 | ASI | 1113.59 | 10 | 114.36 | 5 |

*The doses were calculated based on the respective molecular weight of the free compound and purity.

The test compounds were administered through oral gavage. Oral route of administration is one of the preferred routes of administration in humans.

In accordance with Table 1, the test compound formulations were administered as follows.

Study Group 1: Single dose oral pharmacokinetic study of ResArgin in male Sprague Dawley rats at a dose equivalent to 500 mg of L-Arginine/kg body weight. Male Sprague Dawley Rats aged 8-10 weeks were used for experimentation after a minimum 3 days of acclimatization. Fasted animals were administered ResArgin in a recommended vehicle (0.5% (w/v) Carboxymethylcellulose in sterile water) by oral route with a dose equivalent to 500 mg L-Arginine/kg body weight at dose volume of 10 mL/kg body weight.

Under mild isoflurane anesthesia, blood specimens were collected at time points (0.0 h, 0.16 h, 0.25 h, 0.50 h, 1.00 h, 2.00 h, 4.00 h, 6.00 h) post-dose as mentioned in Table 1 into pre-labeled tubes containing anticoagulant (K2EDTA—2 mg/mL blood) during the next 24 hours. Collected blood specimens were centrifuged at 4000 rpm, 4° C. for 10 minutes and plasma was separated and stored at −80° C. until analysis.

Study Group 2: Single dose oral pharmacokinetic study of L-Arginine in male Sprague Dawley rats at a dose of 500 mg/kg body weight. Male Sprague Dawley Rat aged 8-10 weeks were used for experimentation after a minimum 3 days of acclimatization. Fasted animals were administered L-Arginine in a recommended vehicle (0.5% (w/v) Carboxymethylcellulose in sterile water by oral route with 500 mg/kg body weight at dose volume of 10 mL/kg body weight.

Under mild isoflurane anesthesia, blood specimens were collected at time points (0.0 h, 0.16 h, 0.25 h, 0.50 h, 1.00 h, 2.00 h, 4.00 h, 6.00 h) post-dose as mentioned in Table 1 into pre-labeled tubes containing anticoagulant (K2EDTA—2 mg/mL blood) during the next 24 hours. Collected blood specimens were centrifuged at 4000 rpm, 4° C. for 10 minutes and plasma was separated and stored at −80° C. until analysis.

Study Group 3: Single dose oral pharmacokinetic study of L-Arginine HCl in male Sprague Dawley rats at a dose equivalent to 500 mg of L-Arginine/kg body weight. Male Sprague Dawley Rats aged 8-10 weeks were used for experimentation after a minimum 3 days of acclimatization. Fasted animals were administered L-Arginine HCl in a recommended vehicle (0.5% (w/v) Carboxymethylcellulose in sterile water) by oral route with a dose equivalent to 500 mg L-Arginine/kg body weight at dose volume of 10 mL/kg body weight.

Under mild isoflurane anesthesia, blood specimens were collected at time points (0.0 h, 0.16 h, 0.25 h, 0.50 h, 1.00 h, 2.00 h, 4.00 h, 6.00 h) post-dose as mentioned in Table 1 into pre-labeled tubes containing anticoagulant (K2EDTA—2 mg/mL blood) during the next 24 hours. Collected blood specimens were centrifuged at 4000 rpm, 4° C. for 10 minutes and plasma was separated and stored at −80° C. until analysis.

Study Group 4: Single dose oral pharmacokinetic study of Arginine Silicate Inositol (ASI) in male Sprague Dawley rats at a dose equivalent to 500 mg of L-Arginine/kg body weight. Male Sprague Dawley Rats aged 8-10 weeks were used for experimentation after a minimum 3 days of acclimatization. Fasted animals were administered ASI in a recommended vehicle (0.5% (w/v) Carboxymethylcellulose in sterile water) by oral route with a dose equivalent to 500 mg L-Arginine/kg body weight at dose volume of 10 mL/kg body weight.

Under mild isoflurane anesthesia, blood specimens were collected at time points (0.0 h, 0.16 h, 0.25 h, 0.50 h, 1.00 h, 2.00 h, 4.00 h, 6.00 h) post-dose as mentioned in Table 1 into pre-labeled tubes containing anticoagulant (K2EDTA—2 mg/mL blood) during the next 24 hours. Collected blood specimens were centrifuged at 4000 rpm, 4° C. for 10 minutes and plasma was separated and stored at −80° C. until analysis.

All test animals were observed for any apparent signs of toxicity related to dose or test item during the study period.

Data Analysis and Results of Testing

Bioanalytical method(s) for the determination of arginine in Rat plasma were developed using LC-MS/MS. The developed methods were used for plasma sample analysis. Chromatograms were acquired using Analyst® software version 1.4.2. The concentration of the unknown sample was calculated from the following equation using regression analysis with peak area ratio of standard and ISD vs calibration standards concentration, using weighting factor (1/x²) in accordance with Equation (1):

y=mx+c Equation (1)

Wherein, x=concentration of drug;

m=slope of calibration curve;

y=peak area ratio;

c=intercept of the calibration curve; and

1/x² was used as the weighting factor.

The pharmacokinetic parameters including Cₘₐₓ, Tₘₐₓ, AUC₀₋ₜ, AUC_{0-inf}, T_{1/2} and MRTₗₐₛₜ of the arginine in rat plasma were determined from the concentration time data by using non-compartmental analysis (Phoenix WinNonlin 6.3, Pharsight Corporation, Princeton, N.J., USA).

Preparation of reagents and stock solutions were performed in the standard manner, in accordance with known methods. Preparation of spiked CC standard and QC samples in plasma were performed in the standard manner. Extraction of blood samples and LC/MS were performed in the standard manner. See, for example, Cox, K. A., et al., “Novel in vivo procedure for rapid pharmacokinetic screening of discovery compounds in rats,” Drug Discovery Today, May 1999; 4:232-237. See also, PCSEA guidelines for Laboratory Animal Facility, Indian Journal of Pharmacology (2003) 35: 257-274. The following references contain appropriate protocols for measuring the levels of the components of ASI, namely arginine, silica, and inositol. For arginine: R. Schuster, “Determination of Amino Acids in Biological, Pharmaceutical, Plant, and Food Samples by Automated Percolumn Derivatization and HPLC”, Journal of Chromatography (1988) 431: 271-284; and Henderson, J. W., Ricker B. D., Bidlingmeyer, B. A., and Woodward, C., “Rapid, Accurate, Sensitive, and Reproducible HPLC Analysis of Amino Acids, Amino Acid Analysis Using Zorbax Eclipse-AAA columns and the Agilent 1100 HPLC,” (Agilent Publications, 2000)./ For silica/silicate: Perkin-Elmer, “Analytical Methods for Atomic Absorption Spectrophotometry,” Norwalk Conn. (2000). For inositol: Tagliaferri, E. G., Bonetti, G., and Blake, C. J., “Ion Chromatographic Determination of Inositol in Infant Formulae and Clinical Products For Enteric Feeding,” J Chromatogr A. (2000) May 26; 879(2): 129-135.

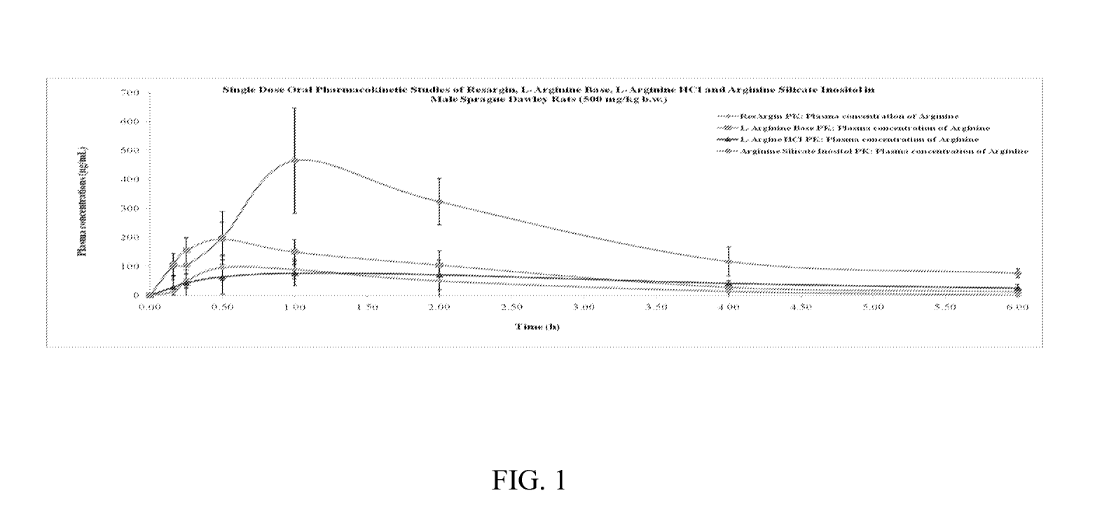

The results of the single dose oral pharmacokinetic study from testing in fasted male Sprague Dawley rates (equivalent dose of 500 mg/kg L-Arginine) for L-Arginine (free base), L-Arginine HCl, ResArgin, and Arginine silicate inositol (ASI), measured in plasma conc. of arginine (μg/ml) over time, are shown in FIG. 1. Comparative plasma concentrations of arginine (μg/mL) based on administration of the test compounds demonstrated that both AUC_{inf} and Cₘₐₓ were increased substantially for ASI compared with Arginine base, 3-fold and 2.35-fold, respectively. Stated in another way, AUC_{inf} for ASI increased by 209% over Arginine base in plasma, while Cₘₐₓ for ASI increased by 135% over Arginine base in plasma.

Comparative plasma mean PK parameters of Arginine are shown in Table 2 for the test compounds.

TABLE 2

| | | | | Arginine |
|---|---|---|---|---|
| | | L-Arginine | L-Arginine | Silicate Inositol |
| PK Parameter | ResArgin PK | Base PK | HCl PK | PK |
| Dose | 500 | 500 | 500 | 500 |
| (mg/kg b.w.) | | | | |
| Cₘₐₓ (μg/mL) | 108.988 ± 42.180 | 201.500 ± 54.2880 | 94.650 ± 26.2010 | 474.300 ± 169.1040* |
| Tₘₐₓ (hr) | 0.800 ± 0.2740 | 0.650 ± 0.3350 | 1.100 ± 0.5480 | 1.100 ± 0.5480 |
| AUCₗₐₛₜ | 197.510 ± 124.2100 | 445.405 ± 106.3480 | 305.775 ± 92.7140 | 1248.990 ± 276.1000* |
| (hr * μg/mL) | | | | |
| AUC_{inf} | 234.492 ± 170.4810 | 485.881 ± 113.5250 | 442.336 ± 122.1220 | 1499.898 ± 233.6230* |
| (hr * μg/mL) | | | | |
| AUC_{% extrap}(%) | 13.014 ± 11.3050 | 8.195 ± 5.9280 | 27.645 ± 23.5890 | 16.987 ± 10.5790 |
| T_{1/2} (hr) | 1.075 ± 0.4840 | 1.406 ± 0.5100 | 3.080 ± 2.0820 | 2.159 ± 0.8280 |
| MRTₗₐₛₜ (hr) | 1.291 ± 0.3740 | 1.658 ± 0.3400 | 2.479 ± 0.3680 | 2.220 ± 0.2450* |

*p < 0.05 (for ASI vs. L-Arginine base)

Statistical analysis of PK parameters was performed by one way ANOVA followed by Dunnett's Post Test (L-Arginine Base vs ResArgin, L-Arginine HCl, and Arginine Silicate Inositol, respectively).

As shown in Table 2 (and FIG. 1), both AUC_{inf} and Cₘₐₓ of arginine were increased substantially for ASI compared with Arginine base. Notably, each of Cₘₐₓ (μg/mL), AUCₗₐₛₜ (hr*μg/mL), AUCinf (hr*μg/mL), and MRTₗₐₛₜ (hr) demonstrated significance at p<0.05 in comparing ASI with Arginine base using Dunnett's Multiple Comparison Test.

Human Clinical Study

In accordance with the present methods, a human clinical study was performed using ASI, demonstrating that ingestion leads to significant increases in both plasma arginine and serum silica for up to three hours after use (for arginine). See, Kalman D, Feldman S, Samson A, Krieger D., “A clinical evaluation to determine the safety, pharmacokinetics and pharmacodynamics of an inositol-stabilized arginine silicate dietary supplement in healthy adult males,” The FASEB Journal (April 2014) 28(1 Suppl.): LB418.

In addition, the use of ASI at 1,500 mg/day for two weeks led to significant increases in plasma nitric oxide levels which would theoretically have a beneficial effect on circulatory status and increase blood supply to the wound.

Clinical Study Design

Purpose: The purpose of this study was to characterize the PK/PD of Nitrosigine®, an inositol-stabilized arginine silicate dietary supplement.

Type: Prospective, pharmacokinetic (PK)/pharmacodynamic (PD)/safety clinical trial.

Study Group: 10 healthy males, aged 18 to 40 years, with BMI≧18 to <30 kg/m², who do not smoke.

Dosing: Nitrosigine® was administered as daily doses of 1,500 mg (3×500 mg tablets). Study subjects were supplied with ASI tablets and instructed to take 3×500 mg tablets with 8 ounces of water at the first treatment visit (Visit 2) and then continue taking this dose daily for 14 days.

3 scheduled study visits:

Baseline (Visit 1): screening tests

Visit 2: single dose evaluation (0-6 hrs), 1500 mg—this was also utilized as the first dose of the 14 day study. It is noted in addition that Visit 2 is also the beginning of the 14 day study, during which subjects take product once a day for 14 days.

Visit 3: 14 days later, single dose (0-6 hrs)

All 10 subjects finished the 14-day study. No adverse events related to the study medication.

PK Measurements: Plasma arginine and serum silicon levels were measured after the first dose on Day 1 and again after a dose taken on Day 14, after 14 days of dosing. Arginine absorption: Significant increase after 0.5 hr. (P=0.002); Tₘₐₓ Day 1: 1.05±0.50 hr. Silicon absorption: Significant increase after 1 hr. (P=0.002); Tₘₐₓ Day 1: 2.15±1.75 hr.

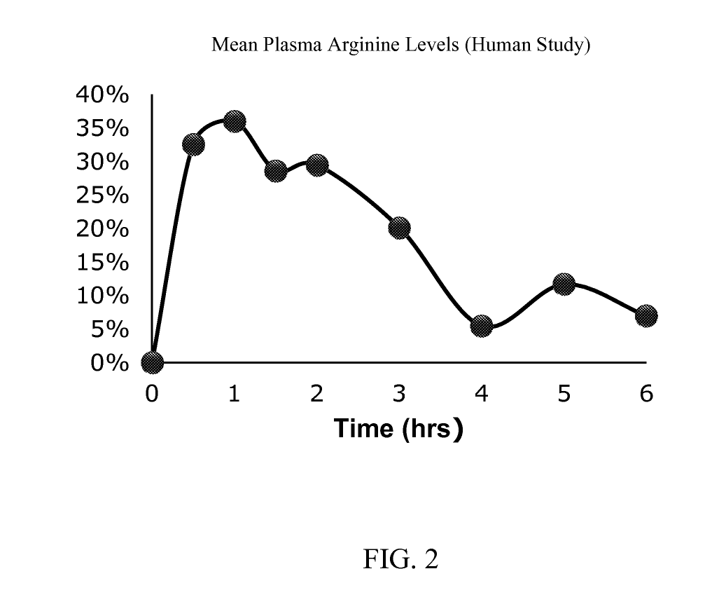

In conclusion, Nitrosigine® was demonstrated to be a bioavailable source of arginine and silicon. Nitrosigine® significantly increased plasma arginine levels in 30 minutes (showing an increase of greater than about 30% plasma concentration relative to baseline). A single dose of Nitrosigine® significantly raised blood arginine and silicon levels for up to 3 hours. For example, after 1 hour mean plasma arginine was found to be increased by greater than 35% relative to baseline. As shown in FIG. 2, ASI supplementation resulted in statistically significant increases in plasma arginine from baseline at 0.5, 1, 1.5, 2, 3, and 5 hours (P<0.05) in human subjects.

Nitric oxide (NO) levels were measured in each study subject before, and at a number of time points after, administration of ASI as a single dose and after 14 days of daily dosing. NO was measured using salivary test strips, using a color scale for “Depleted”, “Low”, and “Normal” levels. There was a significant change in NO levels between the first treatment visit (Visit 2) and after 14 days (Visit 3). Six subjects who were “Depleted” at Visit 2 increased to “Low” at Visit 3, while no subjects went from “Low” at Visit 2 to “Depleted” at Visit 3. In addition, the Visit 3 treatment after 1 hr gave 3 subjects “Depleted”, 4 subjects “Low”, and 3 subjects “Normal.” This “asymmetric” change pattern was statistically significant (p=0.031).

Thus, ASI supplementation increased NO levels with a significant increase seen after 14 days of administration.

It has also been shown that ASI treatment significantly enhances blood proteins related to vasodilation and heart health.

In one embodiment, it has been shown using standard gene expression methods in proteomics that expression levels of certain proteins implicated in wound healing processes were increased. For example: Alpha-2-antiplasmin which is a key component in facilitating blood clotting (+40%; p=0.0005); Fibrinogen gamma chain which acts as a cofactor in platelet aggregation (+70%; p=0.0005). Thus, protein levels via measurement of induction of gene expression may be measured.

It is believed that further studies in which ASI is used in place of arginine in current clinical nutrition products would show an additive effect of the arginine and silica compared to just arginine alone, and at lower doses.

It is further hypothesized that ASI in combination with additional ingredients can provide beneficial and synergistic effects with respect to wound healing and its associated outcomes.

The composition includes an effective amount of arginine silicate inositol (ASI) one or more nutritional components, flavoring compounds, dietary supplements, or other edible components. Useful components include vasodilators, NO-precursors, or eNOS activators. Further optional components can include compounds selected from proline, hydroxyproline, IGF-1 activators, or polyamine precursors.

The optional components can include compounds selected from vasodilators, NO-precursors, or eNOS activators, and IGF-1 activators, such as, but not limited to: citrus bioflavonoids (quercetin, rutin, isoquercetin, synephrine, octopamine, and the like); CoQ10, thiamine, citrulline malate, nicotinamide adenoside dinucleotide (NAD), nicotinamide riboside (NR), citrulline, resveratrol-arginine conjugate (ResArgin), lutein, lycopene, capsaicin, arginine alpha ketoglutarate (Arginine AKG), L-arginine pyroglutamate, arginine ketoisocaproate, ornithine alpha ketoglutarate (Ornithine AKG), omega-3 fatty acids (DHA, EPA, and the like), L-norvaline, caffeic acid, nitrate, taurine, arginine ethyl ester, carnosine, vanadyl sulfate, L-alpha glycerophosphorylcholine (Alpha GPC), Pinus pinaster (Pycnogenol®), turmeric (curcumin, demethoxycurcumin, bis-demethoxycurcumin, and the like), rutacaerpine, Epimedium spp., garlic (allicin, alliin, and the like), flaxseed, flaxseed ligans (alpha linolenic acid (ALA), gamma linolenic acid (GLA)), Schisandria (Schisandrin, Schisandrol A and B, Gamma schisandrin, and the like), green tea catechins (catechin, ECGC, ECG, EGC, and the like), black tea, dong quai (ligustilide), Andrographis (Andrographolides), grape extract (resveratrol, and the like), anthocyanins (cyanidin 3-glucoside (C3G), cyanidin 3-rutinoside, delphinidin 3-glucoside, malvidin 3-glucoside, and the like), Danshen, Beta vulgaris root, celery (3-N-butylphthalide), Berberine, Feverfew, Jasmine, Lemon balm, vinpocetine, Lotus, White horehound, Lemongrass, Yerba mate, Peony, Mustard, Motherwort, Cramp bark, Grapeseed, Proanthocyanidins (PACs, including Procyanidin A1, Procyanidin A2, Procyanidin B1, Procyanidin B2, and the like), Spinach (containing nitrates), Kale (containing nitrates), Broccoli (containing nitrates), Beet (containing nitrates), Chocolate (Cocoa flavonoids, theobromine, theophylline, phenylethylamine (PEA), and the like), Hawthorn, Hawthorn flavonoids (hyperoside, vitexin, isovitexin, and the like), Catauba extract, apple polyphenols, and combinations thereof.

Useful pharmaceutical vasodilators include, but are not limited to, NO-inducers such as: glyceryl trinitrate (a.k.a. nitroglycerin), isosorbide mononitrate, isosorbide dinitrate, clonitrate, ettriol trinitrate (ETTN), erythrityl tetranitrate, pentaerythritol tetranitrate (PETN), pentrinitrol, D-mannitol hexanitrate, trolnitrate phosphate, sodium nitroprusside, PDE5 inhibitors (slidenafil, tadalafil, vardenafil), papaverine, bamethan, bencyclane, beraprost, betahistine, brovincamine, bufeniode, buflomedil, butalamine, cetiedil, chromonar, ciclonicate, cinepazide, cinnarizine, clobenfurol, cloricromen, cyclandelate, dilazep, droprenilamine, eburnamonine, efloxate, eledoisin, etafenone, fasudil, fendiline, fenoxedil, flunarizine, hexobendine, ibudilast, ifenprodil, iloprost, inositol niacinate, itramin tosylate, kallidin, kallikrein, khellin, lidoflazine, lomerizine, moxisylyte, nafronyl, nicotinyl alcohol, nimodipine, nylidrin, pentifylline, pimefylline, piribedil, trapidil, trimetazidine, vincamine, vinpocetine, viquidil, visnadine, xanthinol niacinate, bendazol, floredil, medibazine, tinofedrine, amotriphene, benfurodil hemisuccinate, hepronicate, hepronicate, nicofuranose, suloctidil, or salts and/or prodrugs thereof.

Useful IGF-1 activators include, but are not limited to, tabimorelin, capromorelin, ibutamoren, and the like.

In one embodiment, ASI can be provided in daily dosages of from about 5 mg to about 25 g, in particular in a human patient, for example. Another suitable daily dosage is from about 50 mg to about 25 g, in particular in a human patient. Another suitable daily dosage is from about 10 mg to about 10 g, in particular in a human patient. Another suitable dosage range is from about 500 mg to about 3 g daily. Another suitable dosage is about 750 mg administered twice daily (1,500 mg total). The daily dose of ASI may be administered in one or more unit dosages or subdivided unit dosages, as needed. The daily dose of ASI may be administered in one or more servings. For example, a useful dose is about 1500 mg of ASI, which can be provided in one or more servings, for example, in one or more tablets or drink powders (reconstitutable or ready-to-drink, i.e. RTD).

In an embodiment, the daily dosage of ASI in a human subject can range up to 25 g, with a preferred daily dosage of between about 1 g to about 3 g, taken in one or more servings per day.

The nutritional compositions and/or nutraceutical compositions of the present invention may be administered in combination with a nutraceutically acceptable carrier. The active ingredients in such formulations may comprise from 1% by weight to 99% by weight, or alternatively, 0.1% by weight to 99.9% by weight. “Nutraceutically acceptable carrier” means any carrier, diluent or excipient that is compatible with the other ingredients of the formulation and not deleterious to the user. Useful excipients include microcrystalline cellulose, magnesium stearate, calcium stearate, any acceptable sugar (e.g., mannitol, xylitol), and for cosmetic use an oil-base is preferred.

The pharmaceutical compositions (including topical formulations) of the present invention may be administered in combination with a pharmaceutically acceptable carrier. The active ingredients in such formulations may comprise from 1% by weight to 99% by weight, or alternatively, 0.1% by weight to 99.9% by weight. “Pharmaceutically acceptable carrier” means any carrier, diluent or excipient that is compatible with the other ingredients of the formulation and not deleterious to the user.

An individual subject may be an animal or a human. Animal subjects include large domestic mammals, for example, cattle (or other bovine species), horses, pigs, sheep, goats, other livestock, and the like. Animal subjects may also include smaller domestic mammals, such as, but not limited to, dogs, cats, rabbits, and rodents including rats, mice, hamsters, gerbils, guinea pigs, and the like.

Delivery System

Suitable dosage forms include tablets, capsules, solutions, suspensions, powders, gums, and confectionaries. Sublingual delivery systems include, but are not limited to, dissolvable tabs under and on the tongue, liquid drops, and beverages. Edible films, hydrophilic polymers, oral dissolvable films or oral dissolvable strips can be used. Other useful delivery systems comprise oral or nasal sprays or inhalers, and the like.

For oral administration, ASI (optionally combined with other components) may be combined with one or more solid inactive ingredients for the preparation of tablets, capsules, pills, powders, granules or other suitable dosage forms. For example, the active agent may be combined with at least one excipient such as fillers, binders, humectants, disintegrating agents, solution retarders, absorption accelerators, wetting agents, absorbents, or lubricating agents. Other useful excipients include magnesium stearate, calcium stearate, mannitol, xylitol, erythritol, maltodextrin, sweeteners, starch, carboxymethylcellulose, microcrystalline cellulose, silica, gelatin, silicon dioxide, and the like.

The components of the invention, together with a conventional adjuvant, carrier, or diluent, may thus be placed into the form of pharmaceutical compositions and unit dosages thereof. Such forms include solids, and in particular tablets, filled capsules, powder and pellet forms, and liquids, in particular aqueous or non-aqueous solutions, suspensions, emulsions, elixirs, and capsules filled with the same, all for oral use, suppositories for rectal administration, and sterile injectable solutions for parenteral use. Such pharmaceutical compositions and unit dosage forms thereof many comprise conventional ingredients in conventional proportions, with or without additional active compounds or principles, and such unit dosage forms may contain any suitable effective amount of the active ingredient commensurate with the intended daily dosage range to be employed.

The components of the present invention can be administered in a wide variety of oral and parenteral dosage forms. It will be obvious to those skilled in the art that the following dosage forms may comprise, as the active component, either a chemical compound of the invention or a pharmaceutically acceptable salt of a chemical compound of the invention.

For preparing pharmaceutical compositions from a chemical compound of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, solubilizers, lubricants, suspending agents, binders, preservatives, tablet disintegrating agents, or an encapsulating material.

In powders, the carrier is a finely divided solid, which is in a mixture with the finely divided active component. In tablets, the active component is mixed with the carrier having the necessary binding capacity in suitable proportions and compacted in the shape and size desired.

The powders and tablets may contain from five or ten to about seventy percent of the active compound(s). Suitable carriers are magnesium carbonate, magnesium state, talc, sugar, lactose, pectin, dextrin, starch, gelatin, tragacanth, methylcellulose, sodium carboxymethlycellulose, a low melting wax, cocoa butter, and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, are included. Tablets, powders, capsules, pills, cachets, and lozenges can be used as solid forms suitable for oral administration.

Extended-release, sustained-release, and time-release dosage forms are contemplated, as are well-known in the art. In the present disclosure, co-crystals may be optionally used as an adjunct to solid tablet or solid and/or liquid capsule formation, thus providing built-in “extended release” properties based on the components' crystal structure.

Liquid preparations include solutions, suspensions, and emulsions, for example, water or water-propylene glycol solutions. For example, parenteral injection liquid preparations can be formulated as solutions in aqueous polyethylene glycol solution. The chemical compound according to the present invention may thus be formulated for parenteral administration (e.g. by injection, for example bolus injection or continuous infusion) and may be presented in unit dose for in ampoules, pre-filled syringes, small volume infusion or in multi-dose containers with an added preservative. The compositions may take such forms as suspensions, solutions, or emulsions in oily or aqueous vehicles, and may contain formulation agents such as suspending, stabilising and/or dispersing agents. Alternatively, the active ingredient may be in powder form, obtained by aseptic isolation of sterile solid or by lyophilization from solution, for constitution with a suitable vehicle, e.g., sterile, pyrogen-free water, before use.

Aqueous solutions suitable for oral use can be prepared by dissolving the active component in water and adding suitable colorants, flavors, stabilizing and thickening agents, as desired. Aqueous suspensions suitable for oral use can be made by dispersing the finely divided active component in water with viscous material, such as natural or synthetic gums, resins, methylcellulose, sodium carboxymethylcellulose, or other well known suspending agents.

Compositions suitable for topical administration in the mouth includes lozenges comprising the active agent in a flavored base, usually sucrose and acacia or tragacanth; pastilles comprising the active ingredient in an inert base such as gelatin and glycerine or sucrose and acacia; and mouthwashes comprising the active ingredient in suitable liquid carrier.

In accordance with certain embodiments, the cosmetic and/or topical pharmaceutical compositions disclosed herein can be provided in the form of an ointment, cream, lotion, gel or other transdermal delivery systems as described in L. V. Allen, Jr., et al., Ansel's Pharmaceutical Dosage Forms and Drug Delivery Systems, 9^{th} Ed., pp. 272-293 (Philadelphia, Pa.: Lippincott Williams & Wilkins, 2011) which is incorporated herein by reference.

Ointments, as used herein, refer to semi-solid preparations including an ointment base having one or more active ingredients incorporated or fused (i.e., melted together with other components of the formulation and cooled with constant stirring to form a congealed preparation) therein. The ointment base may be in the form of: an oleaginous or hydrocarbon base (e.g., petrolatum or a petrolatum/wax combination); an absorption base which permits the incorporation of aqueous solution resulting in the formation of a water-in-oil emulsion (e.g., hydrophilic petrolatum) or which is a water-in-oil emulsion that permits the incorporation of additional quantities of aqueous solutions (e.g., lanolin); a water-removable base which are oil-in-water emulsions that may be diluted with water or aqueous solutions (e.g., hydrophilic ointment, USP); or a water-soluble base that do not contain oleaginous components (e.g., polyethylene glycol (PEG) formulations which combine PEGs having an average molecular below 600 with a PEG having an average molecular weight above 1,000); and the like.

Creams, as used herein, refer to semisolid preparations containing one or more active or medicinal agent dissolved or dispersed in either a water-in-oil emulsion or an oil-in-water emulsion or in another type of water-washable base. Generally, creams are differentiated from ointments by the ease with which they are applied/spread onto a surface such as the skin and the ease with which they are removed from a treated surface.

Lotions, as used herein, refer to suspensions of solid materials in an aqueous vehicle. Generally, lotions have a non-greasy character and increased spreadability over large areas of the skin than ointments, creams, and gels.

Gels, as used herein, refer to semisolid systems including a dispersion of small and/or large molecules in an aqueous liquid vehicle which is rendered jellylike by the addition of a gelling agent. Suitable gelling agents include, but are not limited to, synthetic macromolecules (e.g., carbomer polymers), cellulose derivatives (e.g., carboxymethylcellulose and/or hydroxypropyl methylcellulose), and natural gums (e.g., tragacanth gum, carrageenan, and the like). Gel preparations may be in the form of a single-phase gel in which the active or medicinal ingredients are uniformly dispersed throughout the liquid vehicle without visible boundaries or a two-phase gel wherein flocculants or small distinct particles of the active or medicinal ingredient are dispersed within the liquid vehicle.

Transdermal preparations may be formed from an ointment, cream, or gel that has been combined with a penetration enhancer and are designed to deliver an active or medicinal ingredient systemically. Penetration enhancers include, for example, dimethyl sulfoxide, ethanol, propylene glycol, glycerin, PEG, urea, dimethyl acetamide, sodium lauryl sulfate, poloxamers, Spans, Tweens, lecithin, and/or terpenes amongst others.

Other suitable semi-solid forms for use as cosmetic and/or topical pharmaceutical compositions include pastes (preparations containing a larger proportion of solid material rendering them stiffer than ointments) and glycerogelatins (plastic masses containing gelatin, glycerin, water, and an active or medicinal ingredient).

In other embodiments the topical and/or cosmetic compositions can be prepared in accordance with dosage forms as described in Sample Preparation of Pharmaceutical Dosage Forms, B. Nickerson, Ed. (New York: Springer, 2011) herein incorporated by reference.

Solutions or suspensions are applied directly to the nasal cavity by conventional means, for example with a dropper, pipette or spray. The compositions may be provided in single or multi-dose form. In compositions intended for administration to the respiratory tract, including intranasal compositions, the compound will generally have a small particle size for example of the order of 5 microns or less. Such a particle size may be obtained by means known in the art, for example by micronization.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, cachet, or lozenges itself, or it can be the appropriate number of any of these in packaged form.

Tablets, capsules and lozenges for oral administration and liquids for oral use are preferred compositions. Solutions or suspensions for application to the nasal cavity or to the respiratory tract are preferred compositions. Transdermal patches comprising creams, lotions, ointments, or salves, and/or other delivery vehicles or delivery systems, for topical administration to the epidermis are preferred. Generally, creams, lotions, ointments, or salves comprising other delivery vehicles or excipients and/or delivery systems for topical delivery are preferred. Also contemplated are microemulsions and gel formulations for topical administration.

A bioactive-containing or drug-containing patch, mesh or stent, or the like, can be used for administration of ASI, for example in delayed release and/or extended release to the body. As used herein, the term “mesh” refers to a mesh, pouch, bag, covering, shell, skin or receptacle comprised of a solid or semi-solid material. A mesh in accordance with the invention is any web or fabric with a construction of knitted, braided, woven or non-woven filaments or fibers that are interlocked in such a way to create a fabric or a fabric-like material that includes a matrix of filaments that define multiple pores. Said mesh can contain a bioactive material such as ASI, which can be incorporated, saturated into, sprayed, impregnated, or applied to the mesh in the standard manner. The ASI-containing mesh may be applied topically to the skin or installed within the body, for example in proximity to a wound, or directly adjoining or contacting the wound.

Further details on techniques for formulation and administration may be found in the latest edition of Remington's Pharmaceutical Sciences (Mack Publishing Co., Easton, Pa.).

Routes of Administration

Generally, the nutritional compositions containing ASI are ingestible. The compounds may be administered by any route, including but not limited to oral, sublingual, buccal, ocular, pulmonary, rectal, and parenteral administration, or as an oral or nasal spray (e.g. inhalation of nebulized vapors, droplets, or solid particles). Parenteral administration includes, for example, intravenous, intramuscular, intraarterial, intraperitoneal, intranasal, intravaginal, intravesical (e.g., to the bladder), intradermal, transdermal, topical, or subcutaneous administration. Also contemplated within the scope of the invention is the instillation of ASI in the body of the patient in a controlled formulation, with systemic or local release of the drug to occur at a later time. For example, the drug may be localized in a depot for controlled release to the circulation, or for release to a local site.

Pharmaceutical compositions of the invention may be those suitable for oral, rectal, bronchial, nasal, pulmonal, topical (including buccal and sub-lingual), transdermal, vaginal or parenteral (including cutaneous, subcutaneous, intramuscular, intraperitoneal, intravenous, intraarterial, intracerebal, intraocular injection or infusion) administration, or those in a form suitable for administration by inhalation or insufflations, including powders and liquid aerosol administration, or by sustained release systems. Suitable examples of sustained release systems include semipermeable matrices of solid hydrophobic polymers containing the compound of the invention, which matrices may be in form of shaped articles, e.g. films or microcapsules.

Without intending to be bound by theory, ASI dosage (and the dosage of other active components) can be estimated or translated from dosages used in animal studies. Doses from animal studies were translated to human doses by utilizing a Kₘ factor ratio, where Kₘ factors were assigned to each animal model based on their body surface area (Reagan-Shaw, et al., FASEB J. (2007) 22:659-661). The human equivalent dose (HED) is equal to the animal dose multiplied by the ratio: animal's Kₘ/human Kₘ.

The treatment may be carried out for as long a period as necessary, either in a single, uninterrupted session, or in discrete sessions. The treating physician will know how to increase, decrease, or interrupt treatment based on patient response. According to one embodiment, treatment is carried out for from about four to about twelve weeks. The treatment schedule may be repeated as required.

Nutritional compositions are prepared as described herein. Dietary supplement compositions are prepared as described herein. The compositions may be used by human consumers or in animal health. In one embodiment, treatment schedule is 1-3 times daily for an undefined period of time in order to achieve improvements in wound healing or repair, for example.

It is understood that certain standard study methods are available to measure the rate of wound healing using ASI, or other parameters indicative of wound healing or repair. Methods for measuring the rate of wound healing are known in the art and include, for example, observing increased epithelialization and/or granulation tissue formation, or lessening of the wound diameter and/or depth. Increased epithelialization can be measured by methods known in the art such as by, for example, the appearance of new epithelium at the wound edges and/or new epithelial islands migrating upward from hair follicles and sweat glands. In addition, there is a new technology called Silhouette (http://www.aranzmedical.com/; 2014 ARANZ Medical Limited, Christchurch, New Zealand). Silhouette is a 3D measurement, imaging and documentation system that provides precise measurement and healing trends along with comprehensive wound surveillance support.

The methods described above may be further understood in connection with the following Examples including clinical studies to measure wound healing.

### Example 1

Established protocols are used for assessment of oral ASI administration for treating wounds, for example, such as used in Kirk, et al., Surgery (1993) 114(2): 155-160.

The Kirk, et al. protocol is used as follows. Thirty healthy, human volunteers (15 men and 15 women) receive daily supplements of ASI (1500 mg) for 2 weeks. Fifteen volunteers receive a placebo tablet. Fibroplastic wound responses are assessed by inserting a polytetrafluoroethylene catheter subcutaneously into the right deltoid region. Epithelialization is examined by creating a 2×2 cm split thickness wound on the lateral aspect of the upper thigh. Mitogenic response of peripheral blood lymphocytes to concanavalin A, phytohemagglutinin, pokeweed mitogen, and allogeneic stimuli is assayed at the beginning and end of supplementation. Polytetrafluoroethylene catheters were analyzed for alpha-amino nitrogen (assessment of total protein accumulation), hydroxyproline (index of reparative collagen synthesis), and DNA accumulation (index of cellular infiltration).

It is expected that after 2 weeks the rate of epithelialization of the skin defect will be significantly enhanced in those subjects treated with ASI in comparison to placebo. It is further expected that the rate of wound healing will be increased in those subjects treated with ASI in comparison to placebo.

Secondary endpoints: It is expected that wound catheter hydroxyproline accumulation and total protein content will increase in those subjects treated with ASI in comparison to placebo. It is further expected that serum insulin-like growth factor-1 (IGF-1) levels will be significantly elevated in the ASI-treated group.

### Example 2

Established protocols are used for assessment of topical ASI administration to wounds, for example, such as those used in J. Burn Care Res. (2009) May-June; 30(3):417-26, Kirk, et al. (1993), or Vits, et al., Int. Wound 1 (2013) December 30.

The Vits, et al. protocol is used as follows. In 22 healthy men (experimental group, n=11; control group, n=11) wounds are induced by ablative laser on both thighs. Hydrogel (placebo) is applied on one of the two wounds, and Hydrogel (containing ASI) is applied on the wound of the other thigh. Progress in wound healing was documented via planimetry on days 1, 4 and 7 after wound induction. From day 9 onwards wound inspections were performed daily accompanied by a change of the dressing and a new application of the gel. It is expected that more rapid improvement in wound healing will be observed in those wounds treated with the ASI-containing gel, with regard to duration or process of wound healing, either by intraindividual or by interindividual comparisons. In accordance with the study protocol no expectation-induced placebo effect is observed on the healing process of experimentally induced wounds in healthy volunteers.

### Example 3

Clinical Study to measure muscle soreness, muscle damage, and recovery. Crossover PCT.

Dosing: single dose or 4 days use at ASI 750 mg 2×/day.

Study group: n=16 males exercising <150 mins/week.

Secondary endpoints: NO level, blood pressure, heart rate, leg circumference, patient survey/self-assessment, e.g., visual analog scale (VAS) for pain, intramuscular biopsy.

After treatment with ASI 750 mg 2×/day for 14 days with a concomitant standard training regimen including exercising quadriceps to exhaustion, it is expected that each listed measurement parameter will improve, e.g., increase in NO levels. Further, it is expected that measurement of cytokine markers of inflammation will show decrease or improvement.

### Example 4

Tissue remodeling and/or repair. Other studies include: bone mineralization using ASI treatment and measurement of other markers (e.g., collagen formation) after ASI treatment; also, wound healing using ASI treatment and markers of skin and/or tissue repair or remodeling.

In an embodiment, topical and/or oral administration of an ASI-containing composition after about several days (0-7 days) or more (8-14 days) results in increased deposition levels of one or more of the following extracellular matrix proteins in skin: collagen I, collagen III, elastin, fibronectin, proteoglycan (e.g., chondroitin, heparan, keratan, hyaluronan), thrombospondin 1 or 2, SPARC (secreted protein, acidic and rich in cysteine), periotin, or fibulin.

### Example 5

### Wound Healing Models

Skin wound healing is a complex biological process activated by signalling pathways of epithelial and non-epithelial cells, which release a myriad of cytokines and growth factors. Rodent models of full thickness incisions comprise a large portion of preclinical wound healing research. It has also been shown that larger species such as the pig have up to 78% correlation to human wound healing. Therefore, the pig is the ideal species for wound healing studies due to the many similarities between pig and human skin and offers great advantages in wound healing research.

Anatomically, the thickness of the pig epidermis is quite similar to human epidermis, particularly for pigs that are around 20 kg. The dermal-epidermal thickness ratio is quite similar and both human and pig show well-developed rete-ridges, dermal papillary bodies and abundant subdermal adipose tissue. Functionally, pig and human are similar in epidermal turnover time, type of keratinous proteins and lipid composition of the stratum corneum. Additionally, the skin innervation is quite similar from pig to human with C-fiber classes between human and pig correlated in both distribution and axonal excitability changes.

Perhaps one of the most compelling arguments for using pig for wound healing is the similarity in the physiological process through which pig and human heal. Pig and human close partial-thickness wounds primarily through reepithelialization, rather than wound contraction as is the case with small animals such as rodents. Impaired Wound healing can also be evaluated by using a diabetic animal.

Experimental Overview.

Animal strains: mouse, rat or healthy domestic pigs.

Standard assessments: body weights; clinical signs and observations; wound score; photographs; photomicrographs/SEM; gross pathology; histology; open field for locomotor activity; Von Frey pain assessment; PK studies.

### Example 5A

### Wound Healing in Pig

A 10 cm wound is created on the flank of the pig. For studies including pain measurements, only one incision per pig as the intact flank serves as the control for pain measurements. For studies only evaluating wound healing, two incisions per pig may be possible depending on the treatment, therapy or dressing that is applied. The incision is a full-thickness incision including the skin and fascia (of several cm longitudinal). For tests as necessary, the incision can also include the muscle.

Wound inflammation scoring can be carried out (the sums of redness and swelling scores) over time. Alternatively, gross pathology can be determined over time in the wound tissue, for example, removed after termination of the subject. Alternatively, histology of the wound can be performed.

### Example 5B

### Wound Healing in Rodents

Study length: 30 days.

Species/Strain: Balb/c mouse or rat.

Total population: n=80

Population per group: 12-16

Proposed test groups: sham, 4 test compound study groups (as in Table 1, PK study)

Dosing route: systemic (e.g., oral or i.v.); daily

Induction of wound/Inclusion criteria: 2 cm full skin thickness incision is performed along the spine in the upper back of the animal, three hours later the incision becomes elliptic; tear shape wound through all layers of the skin. Animals with wounds width ≧6.5 mm at 3 hours post incision are included in the study.

Body weight of the test animals is tracked and clinical signs are observed daily.

Parameters of the wound healing process:

Wound infliction (day 1-3);

Scab formation (day 4-7);

Scab detachment (day 8-12);

Remodeling (day 12-30).

Wound width is measured at the widest area (middle of the wound) using caliper on study days 1,4,7,9,12,14,16, 20, 25. Blood, plasma and/or serum is collected at various time points for further cytokine analysis.

Groups are sacrificed in cohorts at four different time points, and at each time point, 4 animals are sacrificed: days 2, 5, 10 and 25. The entire wound area is harvested and stored in 4% formalin for histology H&E staining and analysis. Changes in wound width can be measured on each of these days.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

The use of the terms “a,” “an,” “the,” and similar referents in the context of describing the presently claimed invention (especially in the context of the claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. Use of the term “about” is intended to describe values either above or below the stated value in a range of approx. ±10%; in other embodiments the values may range in value either above or below the stated value in a range of approx. ±5%; in other embodiments the values may range in value either above or below the stated value in a range of approx. ±2%; in other embodiments the values may range in value either above or below the stated value in a range of approx. ±1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

We claim:

1. A method for treating a wound or injury in an individual, comprising the steps of administering to the individual afflicted with the wound or injury a composition including an effective amount of arginine silicate inositol (ASI), optionally in combination with another component, and a nutraceutically or pharmaceutically acceptable carrier.

2. The method of claim 1, wherein the composition further comprises at least one other component which is a vasodilator, a NO-precursor, an eNOS activator, or an IGF-1 activator selected from the group consisting of: proline, hydroxyproline, quercetin, rutin, isoquercetin, synephrine, octopamine, resveratrol-arginine (1:1) conjugate (ResArgin), CoQ10, thiamine, citrulline malate, nicotinamide adenoside dinucleotide (NAD), nicotinamide riboside (NR), citrulline, lutein, lycopene, capsaicin, arginine alpha ketoglutarate (Arginine AKG), L-arginine pyroglutamate, arginine ketoisocaproate, ornithine alpha ketoglutarate (Ornithine AKG), DHA, EPA, L-norvaline, caffeic acid, nitrate, taurine, arginine ethyl ester, carnosine, vanadyl sulfate, L-alpha glycerophosphorylcholine (Alpha GPC), Pinus pinaster extract (Pycnogenol®), turmeric, curcumin, demethoxycurcumin, bis-demethoxycurcumin, rutacaerpine, Epimedium spp., garlic, allicin, alliin, flaxseed, alpha linolenic acid (ALA), gamma linolenic acid (GLA), Schisandrin, Schisandrol A and B, Gamma schisandrin, catechin, ECGC, ECG, EGC, green tea, black tea, dong quai (ligustilide), Andrographis (Andrographolides), grape extract, resveratrol, cyanidin 3-glucoside (C3G), cyanidin 3-rutinoside, delphinidin 3-glucoside, malvidin 3-glucoside, Danshen, Beta vulgaris root, celery (3-N-butylphthalide), Berberine, Feverfew, Jasmine, Lemon balm, vinpocetine, Lotus, White horehound, Lemongrass, Yerba mate, Peony, Mustard, Motherwort, Cramp bark, Grapeseed, Procyanidin A1, Procyanidin A2, Procyanidin B1, Procyanidin B2, Spinach, Kale, Broccoli, Beet, Chocolate, theobromine, theophylline, phenylethylamine (PEA), Hawthorn, hyperoside, vitexin, isovitexin, Catauba extract, apple polyphenols, nitroglycerin, isosorbide mononitrate, isosorbide dinitrate, clonitrate, ettriol trinitrate (ETTN), erythrityl tetranitrate, pentaerythritol tetranitrate (PETN), pentrinitrol, D-mannitol hexanitrate, trolnitrate phosphate, sodium nitroprusside, slidenafil, tadalafil, vardenafil, papaverine, bamethan, bencyclane, beraprost, betahistine, brovincamine, bufeniode, buflomedil, butalamine, cetiedil, chromonar, ciclonicate, cinepazide, cinnarizine, clobenfurol, cloricromen, cyclandelate, dilazep, droprenilamine, eburnamonine, efloxate, eledoisin, etafenone, fasudil, fendiline, fenoxedil, flunarizine, hexobendine, ibudilast, ifenprodil, iloprost, inositol niacinate, itramin tosylate, kallidin, kallikrein, khellin, lidoflazine, lomerizine, moxisylyte, nafronyl, nicotinyl alcohol, nimodipine, nylidrin, pentifylline, pimefylline, piribedil, trapidil, trimetazidine, vincamine, vinpocetine, viquidil, visnadine, xanthinol niacinate, bendazol, floredil, medibazine, tinofedrine, amotriphene, benfurodil hemisuccinate, hepronicate, hepronicate, nicofuranose, suloctidil, tabimorelin, capromorelin, ibutamoren, and combinations thereof.

3. The method of claim 1, wherein the effective amount of ASI is present in a total daily dosage of from about 50 mg to about 25 g.

4. The method of claim 1, wherein the effective amount of ASI is present in a total daily dosage of from about 500 mg to about 3 g.

5. The method of claim 1, wherein the composition is administered orally.

6. The method of claim 5, wherein the individual is a human, and wherein the effective amount of ASI is present in a total daily dosage of from about 50 mg to about 25 g.

7. The method of claim 6, wherein plasma L-arginine concentration (μg/mL) is increased by at least 30% about 1 hour after administration.

8. The method of claim 1, wherein the wound or injury is improved by healing or remodeling of tissues.

9. The method of claim 8, wherein the wound is on the skin surface, and wherein the rate of wound healing is increased.

10. The method of claim 1, wherein the composition is administered topically.

11. The method of claim 10, wherein the wound or injury is improved by healing or remodeling of tissues, wherein the wound is on the skin surface, and wherein the rate of wound healing is increased.

12. The method of claim 1, wherein the composition is administered intravenously.
